(19) United States
(12) Patent Application Publication
(10) Pub. No.: US 2011/0112010 A1
(43) Pub. Date: May 12, 2011

(54) OCTANOIC ACID FORMULATIONS AND METHODS OF TREATMENT USING THE SAME

(75) Inventors: Mark Hallett, Bethesda, MD (US); John A. McLane, Warwick, RI (US); Fatta B. Nahab, Gaithersburg, MD (US)

(73) Assignees: The Government of the United States of America, as represented by The National Institutes of Health, Rockville, MD (US); Ariston Pharmaceuticals, Framingham, MA (US)

(21) Appl. No.: 12/867,702

(22) PCT Filed: Feb. 12, 2009
(86) PCT No.: PCT/US2009/000876

§ 371 Date: Dec. 8, 2010

Related U.S. Application Data
(60) Provisional application No. 61/065,993, filed on Feb. 15, 2008.

(51) Int. Cl. A61K 38/16; A61K 31/20; A61K 31/5513; A61K 31/23; A61K 31/7028; A61P 25/16; A61P 5/16; A61P 25/08; A61P 1/16

(52) U.S. Cl. 514/1.1; 514/558; 514/221; 514/552; 514/25

(57) ABSTRACT

The invention features pharmaceutical formulations containing octanoic acid or a salt or ester thereof. The pharmaceutical formulations are useful for the treatment of involuntary tremors.

### BACKGROUND OF THE INVENTION

The invention relates to pharmaceutical formulations containing octanoic acid or a salt or ester thereof. Such pharmaceutical formulations can be used to treat involuntary tremors.

Tremors

Tremors are rhythmic, involuntary muscular contractions characterized by shaking movements that can affect the whole body or particular parts of the body such as the head, hands, fingers, eyelids, vocal cords, trunk, and legs. For example, essential tremor is characterized as a bilateral postural tremor with or without kinetic tremor involving hands or forearms, which is visible and persistent. Tremor involving body parts other than upper limbs may also be present, the tremor may be asymmetrical, and the amplitude may fluctuate. Tremors are a heterogeneous disorder caused by different pathogenic mechanisms. While most people experience a tremor at some time, usually because of fear or excitement, a number of neurological diseases that destroy or modify nerve (including brain) tissue or neural transmission may cause tremor. These include Parkinson's disease and multiple sclerosis. Other causes include stroke or head injury; Wilson's disease, a hereditary disorder in which toxic levels of copper accumulate in the tissues; mercury poisoning; an over-active thyroid gland; and liver encephalopathy. Tremor can also occur as a side effect of drugs. Essential tremors are considered to involve cerebellar and or corticothalamus changes in neural transmission, while the tremors associated with Parkinson's may be considered to be a result of nigrostriatal pathology.

Tremors are classified according to severity, how often they occur, and the type of shaking involved. Tremors can be categorized as postural tremor, unilateral tremor, leg or extremity tremor, rigidity, bradykinesia, rest tremor, gait disturbance, focal tremor, isolated head tremor with abnormal posture (head tilt or turning), and sudden or rapid onset. The severity of tremors varies greatly, and is largely dependent on the underlying condition causing the tremor. For example, certain factors such as stress, normal aging, hypoglycemia, or caffeine can only cause minor tremors, whereas more severe tremors can be associated with neurological disorders such as Parkinson's disease, essential tremor, or stroke. Tremors can occur occasionally (sporadic), temporarily (episodic), or can occur at certain intervals (intermittent), and are generally classified as postural, rest, or action tremor.

Tremors can caused by disease or drugs, or can be hereditary (essential tremor is hereditary). Signs and symptoms depend on tremor type and etiology. The most common tremors and their associated signs and symptoms are:

a) Essential Tremor—This is the most common form of all movement disorders. Essential tremors affect 0.4% of the general population and up to 14% of people 65 years and older. Classical essential tremor is predominantly a postural- or action-type tremor and usually the patient has a positive family history of tremor. The tremor of ET most commonly occurs in the arms, but other regions of the body are involved, especially the head (i.e., neck) and voice, and occasionally the trunk, legs and feet, tongue, and other facial musculature. The frequency of the tremor is usually between 4 Hz and 12 Hz, and is inversely related to age, with older patients generally exhibiting tremor frequencies that are at the lower end of this range. Other associated symptoms can include mild gait difficulty and, as a group, patients with essential tremor have an increased incidence of hearing impairment.

b) Physiological Tremor—This tremor is a very-low-amplitude fine tremor (between 6 Hz and 12 Hz) that is barely visible to the naked eye. It is present in every normal individual during maintaining a posture or movement. Neurologic examination results of patients with physiologic tremor are usually normal.

c) Enhanced Physiologic Tremor—This is a high-frequency, low-amplitude, visible tremor that occurs primarily when a specific posture is maintained. Drugs and toxins induce this form of tremor. The suspected mechanism is mechanical activation at the muscular level. Signs and symptoms of drug toxicity or other side effects can or can not be present.

d) Parkinson's Tremor—This tremor type is a low-frequency rest tremor typically referred to as a pill-rolling tremor. In some patients, postural and action tremor can also occur. Parkinson's tremor usually occurs in association with other symptoms, such as micrographia, slowness of movement (bradykinesia), and rigidity.

e) Cerebellar Tremor—This is a low-frequency (less than 4 Hz) intention tremor that usually occurs unilaterally. Common causes are multiple sclerosis, stroke, and cerebellar injury. Signs and symptoms of cerebellar dysfunction can be present, including ataxia, dysmetria, dysdiadokinesia and dysarthria.

f) Holmes' Tremor—Holmes' Tremor or rubral tremor designates a combination of rest, postural, and action tremors due to midbrain lesions in the vicinity of the red nucleus. This type of tremor is irregular and slow frequency (4.5 Hz). Signs of ataxia and weakness can be present. Common causes include cerebrovascular accident and multiple sclerosis, with a possible delay of 2 weeks to 2 years in tremor onset and occurrence of lesions.

g) Drug-induced Tremor—This type of tremor can occur as a side effect of drugs, including amphetamines, antidepressants, antipsychotics, caffeine, and lithium, and as a result of withdrawal from alcohol or addictive drugs.

h) Tremor Due to Systemic Disease—This tremor usually occurs when the patient is moving or assumes a particular position. Associated symptoms include asterixis, mental status changes, and other signs of systemic illness. Diseases such as thyrotoxicosis and hepatic failure as well as delirium tremens and drug withdrawal are among the common causes.

i) Psychogenic Tremor—This type of tremor can involve any part of the body, but most commonly affects the extremities. Usually, tremor onset is sudden and begins with a combination of postural, action, and resting tremors. Psychogenic tremor decreases with distraction and is associated with multiple other psychosomatic complaints.

j) Orthostatic Tremor—This type is a variant of essential tremor, and occurs in the legs immediately on standing; it is relieved by sitting down. Orthostatic tremor is usually high frequency (14 Hz to 18 Hz), and no other clinical signs and symptoms are present.

Tremors have the potential to interfere with the daily activities of individuals who suffer from them. Most often, fine motor skills are diminished, resulting in difficulties performing everyday tasks such as writing. In addition, tremors can affect the vocal cords, resulting in a shaky or quivering voice, making communication difficult.

Therapeutic Treatments

Current treatments for individuals suffering from tremors can be ineffective with long-term use or produce unwanted side effects. Beta blockers, such as propranolol (commercially sold as Inderal), nadolol, and metoprolol, which are normally used to treat high blood pressure, are often prescribed to treat individuals suffering from tremors. These pharmacological agents block the action of neurotransmitters, particularly compounds related to adrenaline. Possible side effects from these treatments include dizziness, fatigue, nausea, impotence, orthostatic hypotension, depression, confusion, and memory loss. These medications typically are not prescribed to individuals with asthma, diabetes, or certain heart problems.

Anticonvulsants, such as primidone (Mysoline), acetazolamide, methazolamide, valproic acid and gabapentin, can be effective in people who don't respond to beta blockers. As with beta-blockers, these medications modulate the function of some neurotransmitters. Side effects include headaches, sedation, confusion, depression, paresthesias, and gastrointestinal disturbances. In double-blind controlled studies, some of these agents have proved to be no more efficacious than placebo. There is considerable variation amongst patients treated with these beta-blockers or anticonvulsants, and these agents can have limited efficacy in reducing the effects of tremors on fine motor manipulations.

Benzodiazepines such as diazepam (Valium), alprazolam (Xanax), chlordiazepoxide, and clonazepam can improve tremor in some patients with essential tremor. Benefits associated with benzodiazepine therapy in these patients may be due, in part, to its anxiolytic effects. Side effects include excessive sedation, confusion, and memory loss. A number of other agents previously had been tried but showed inconsistent benefit in the treatment of essential tremor. In small trials, the calcium channel blockers nimodipine and nicardipine have shown some promise.

Botulinum toxin types A and B have been used to treat dystonia and spasticity, and are now being used as a therapeutic option for selected patients with tremor. Botulinum toxin acts through presynaptic inhibition of acetylcholine release at the neuromuscular junction. Recently, intramuscular injections of botulinum toxin in the hand have been used to reduce tremor by weakening local muscles.

Parkinson's disease tremor, which is believed to be related to low levels of dopamine in certain parts of the brain, usually improves with dopaminergic and anticholinergic medications. The dopaminergic agents carbidopa and levodopa are often prescribed as a combination first line approach for the treatment of parkinsonian tremor. Levodopa is taken orally and is converted to dopamine in the brain, resulting in increased brain dopamine concentrations. Carbidopa is added to the levodopa to prevent the breakdown of levodopa before it crosses into the brain. This combination medicine was approved by the FDA in 1988.

In addition to levodopa, other dopaminergic agents include pramipexole, ropinirole, pergolide, and amantadine. The combination of dopaminergic agents and anticholinergics is effective in tremor-predominant Parkinson's disease. Anticholinergics include trihexyphenidyl, benztropine, and procyclidine. The potential side effects of this combination dopaminergic-anticholinergic therapy include dry mouth, blurry vision, urinary difficulty, confusion, nausea, hallucinations, insomnia, leg edema, and livedo reticularis and the effectiveness of these drugs to control the tremors is reduced over time.

Surgery can be an option for people whose tremors are severely disabling and don't improve with medications. Surgical management includes ablative therapy through stereotactic thalamotomy or chronic thalamic deep brain stimulation. The ventral intermediate nucleus of the thalamus is the best target for both ablative and deep brain stimulation surgeries. Contraindications for surgical management of essential tremor include unstable medical illnesses, swallowing difficulty, and marked cognitive problems.

Stereotactic thalamotomy involves destroying a tiny part of the thalamus to relieve tremor on the opposite side of the body. The majority of people who undergo the operation experience substantial relief from essential tremor, but the operation as usually performed can relieve tremors only on one side of the body. Operation on both sides of the thalamus poses a risk of irreversible speech loss and negative side effects concerning balance and coordination.

Chronic thalamic deep brain stimulation (DBS) involves implanting a device called a thalamic stimulator. A pacemaker-like chest unit transmits electrical pulses through a wire to a lead implanted in the thalamus. The pulses interrupt signals from the thalamus that help cause tremors. The pulse generator can be turned on and off by passing a magnet over the chest unit. This procedure doesn't pose the risks of thalamotomy and can be performed on both sides of the brain.

One inexpensive agent that has shown promise in treating symptomatic tremors is ethanol, which can reduce tremors by reducing or dampening the synchronized oscillations which cause the tremor. Bain reported that ethanol was an effective treatment in 50% of patients with hereditary essential tremor (Bath et al., Brain 1994, 117: 805-824). However, the effective dose for treatment approaches levels inducing intoxication in patients, limiting its usefulness as a therapeutic agent (Rappaport et al. Life Science 1984, 34: 49-54). In addition, the large doses can result in patients not using the treatment regularly because it interferes with daily activities and can have a social or religious stigma associated with its use (Koller et al., Neurology 1985, 35:1660-2). Alternatively, aliphatic alcohols, other than ethanol, have been used to treat tremors. U.S. Pat. No. 4,897,426 to Llinas et al., entitled “Method for blocking calcium channels”, describes the use of aliphatic alcohols (C₂-C₁₀ alkyl alcohols), octanols in particular, to treat essential, severe essential, physiological, rubral and Parkinson's-associated tremors.

Given the limitations of current treatments for tremors, alternative therapeutic agents are needed.

### SUMMARY OF THE INVENTION

The present invention provides novel pharmaceutical formulations, routes of administration and dosing regimens for octanoic acid and salts or esters thereof.

Accordingly, in a first aspect the invention features a pharmaceutical composition in unit dosage form including octanoic acid, or a salt or ester thereof, in an amount sufficient to treat involuntary tremors when administered to a subject. The unit dosage form can include from 1 mg to 1.0 g, 1 mg to 0.75 g, 1 mg to 0.5 g, 1 mg to 0.25 g, 1 mg to 100 mg, 5 mg to 750 mg, 5 mg to 250 mg, 10 mg to 750 mg, 10 mg to 250 mg, 15 mg to 100 mg, 15 mg to 250 mg, 15 mg to 500 mg, 15 mg to 750 mg, 25 mg to 100 mg, 25 mg to 250 mg, 25 mg to 500 mg, 25 mg to 750 mg, 35 mg to 100 mg, 35 mg to 250 mg, 35 mg to 500 mg, 35 mg to 750 mg, or from 50 mg to 250 mg of octanoic acid, or a salt or ester thereof. In certain embodiments the unit dosage form includes octanoic acid. In other embodiments the unit dosage form includes an ester of octanoic acid (e.g., 1-methyl-1,2-ethanediyl octanoate (1,2-dicaprylin), methyl octanoate, ethyl octanoate, propyl octanoate, butyl octanoate, hexyl octanoate, heptyl octanoate, octyl octanoate, octanoylglucuronide, 1,2,3-propanetriyl octanoate, or any ester of octanoic acid described herein). In still other embodiments the unit dosage form includes a salt of octanoic acid, such as an alkali metal salt, an alkaline earth salt, or a basic addition salt. The unit dosage form can be, for example, a tablet, pill, capsule, or caplet. In certain embodiments, the octanoic acid, or a salt or ester thereof, is substantially pure. In still other embodiments, the unit dosage form further includes a second agent selected from beta blockers, anticonvulsants, benzodiazepines, or Botulinum toxin type A and/or B (the second agent can be any agent described herein).

In a related aspect, the invention features a method of treating an involuntary tremor in a subject in need thereof by administering to the subject a pharmaceutical composition including octanoic acid, or a salt or ester thereof, in an amount sufficient to treat the involuntary tremor. The pharmaceutical composition can be administered, for example, one, two, or three times daily. In certain embodiments the pharmaceutical composition includes octanoic acid. In other embodiments the pharmaceutical composition includes an ester of octanoic acid (e.g., 1-methyl-1,2-ethanediyl octanoate (1,2-dicaprylin), methyl octanoate, ethyl octanoate, propyl octanoate, butyl octanoate, hexyl octanoate, heptyl octanoate, octyl octanoate, octanoylglucuronide, 1,2,3-propanetriyl octanoate, or any ester of octanoic acid described herein). In still other embodiments the unit dosage form includes a salt of octanoic acid, such as an alkali metal salt, an alkaline earth salt, or a basic addition salt. The pharmaceutical composition can be formulated in unit dosage form (e.g., a tablet, pill, capsule, or caplet) or formulated as a syrup, or elixir. In certain embodiments, the octanoic acid, or a salt or ester thereof, is substantially pure. In still other embodiments, the pharmaceutical composition further includes a second agent selected from beta blockers, anticonvulsants, benzodiazepines, or Botulinum toxin type A and/or B (the second agent can be any agent described herein).

The invention further features a kit including (i) a pharmaceutical composition including octanoic acid, or a salt or ester thereof, and (ii) instructions for administering the pharmaceutical composition to a subject for the treatment of involuntary tremors. In certain embodiments the pharmaceutical composition includes octanoic acid. In other embodiments the pharmaceutical composition includes an ester of octanoic acid (e.g., 1-methyl-1,2-ethanediyl octanoate (1,2-dicaprylin), methyl octanoate, ethyl octanoate, propyl octanoate, butyl octanoate, hexyl octanoate, heptyl octanoate, octyl octanoate, octanoylglucuronide, 1,2,3-propanetriyl octanoate, or any ester of octanoic acid described herein). In still other embodiments the unit dosage form includes a salt of octanoic acid, such as an alkali metal salt, an alkaline earth salt, or a basic addition salt. The pharmaceutical composition can be formulated in unit dosage form (e.g., a tablet, pill, capsule, or caplet) or formulated as a syrup, or elixir. In certain embodiments, the octanoic acid, or a salt or ester thereof, is substantially pure. In still other embodiments, the kit further includes a second agent selected from beta blockers, anticonvulsants, benzodiazepines, or Botulinum toxin type A and/or B (the second agent can be any agent described herein).

The methods and kits of the invention can be used to treat involuntary tremor, such as essential tremor, drug-induced tremor, or disease-induced tremor. The involuntary tremor can be drug-induced tremor, such as drug-induced tremor associated with the use of cysclosporine, antidepressants, amphetamines, antipsychotics, or caffeine. In certain embodiments, the involuntary tremor is disease-induced tremor, such as involuntary tremor associated with Parkinson's disease, Multiple Sclerosis, stroke, head injury, Wilson's disease, mercury poisoning, over-active thyroid gland, or liver encephalopathy.

In an embodiment of any of the above-aspects, the octanoic acid, or a salt or ester thereof, can be formulated for delivery by a mechanical device to deliver the formulation over an extended period of time. The device can be, for example, a degradable implant; a transcutaneous patch; a catheter; an implantable pump; a percutaneous pump; an infusion pump; or an iontophoresis device. Mechanical delivery devices can be used alone or in combination with a formulation for controlled, sustained, timed, delayed, or extended release. For example, a transdermal patch can include a permeation enhancer, for example, a glycolipid, a non-esterified fatty acid, an aliphatic alcohol, a fatty acid ester of an aliphatic alcohol, a cyclohexanol, a fatty acid, ester of glycerol, a glycol, or an aliphatic alcohol ether can be used. Other components such as a stabilizer, a solubilizer, a surfactant and a plasticizer can be present in a transdermal patch (see, for example, U.S. Patent Application No. 20020127254).

By “pharmaceutical composition” is meant a composition containing octanoic acid (e.g., 1-octanoic acid, also known as caprylic acid) or a salt or an ester of octanoic acid, formulated with a pharmaceutically acceptable excipient, and manufactured in compliance with the rules of a governmental regulatory agency as part of a therapeutic regimen that includes instructions for the administration of the composition to a subject having tremors or a condition or disease associated with tremors. Pharmaceutical compositions can be formulated, for example, for intravenous administration (e.g., as a sterile solution free of particulate emboli and in a solvent system suitable for intravenous use), or any other formulation described herein. The pharmaceutical compositions of the invention contain octanoic acid, or a salt or an ester of octanoic acid, in an amount sufficient to reduce the frequency, amplitude, or severity of an involuntary tremor in a subject in a clinically relevant manner, or prevent the onset of involuntary tremors in a subject in a clinically relevant manner when administered to a subject.

By “an amount sufficient” is meant the amount of octanoic acid, or a salt or an ester of octanoic acid, required to reduce the frequency, amplitude, or severity of an involuntary tremor in a subject in a clinically relevant manner, or prevent the onset of involuntary tremors in a subject in a clinically relevant manner. A sufficient amount of octanoic acid, or a salt or an ester of octanoic acid, used to practice the present invention for therapeutic treatment of tremors may vary depending upon the manner of administration, the age, body weight, and general health of the patient. Typically an amount sufficient of octanoic acid will be at least 1 mg of octanoic acid. For esters of octanoic acid an amount sufficient will be a mass that yields at least 1 mg of octanoic acid upon complete hydrolysis of the ester. For salts of octanoic acid an amount sufficient will be a mass that yields at least 1 mg of octanoic acid upon complete protonation of the octanoate salt.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

The term “unit dosage forms” refers to physically discrete units suitable as unitary dosages for human subjects, each unit containing a predetermined quantity of octanoic acid, or a salt or ester thereof, in amounts of between 1 milligrams and 2 grams, 25 milligrams and 2 grams, 50 milligrams and 2 grams, or 75 milligrams and 2 grams.

As used herein, the term “substantially pure” refers to a pharmaceutical composition containing octanoic acid, or a salt or ester thereof, as an active ingredient and is less than 5%, 3%, 1%, or even 0.5% by mass other fatty acids or salts or esters of other fatty acids.

As used herein, the term “octanoic acid” refers to compounds having the molecular formula C₇H₁₅COOH.

As used herein, the term “ester” refers to a group of esters of octanoic acid including 1-methyl-1,2-ethanediyl octanoate (1,2-dicaprylin), methyl octanoate, ethyl octanoate, propyl octanoate, butyl octanoate, hexyl octanoate, heptyl octanoate, octyl octanoate, octanoylglucuronide, 1,2,3-propanetriyl octanoate (tricaprylin, trioctanoin) and compounds described by formula I:

[C₇H₁₅—C(O)—O]ₙ—R (I)

In formula I, R is a C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl, and n is an integer from 1 to 10, with the proviso that R is not a therapeutically active substance.

As used herein, a “salt” of octanoic acid refers to an octanoate salt. Octanoate salts that can be used in the methods and compositions of the invention include, without limitation, metal salts, such as sodium, potassium, lithium, magnesium, calcium, zinc, aluminium, and iron; and basic addition salts, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)methylamine (tromethamine) salts.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇.

As used herein, the terms “alkyl” and the prefix “alk-” are inclusive of both straight chain and branched chain groups and of cyclic groups, i.e., cycloalkyl. Exemplary cyclic groups include cyclopropyl, cyclobutyl, cyclopentyl, and cyclohexyl groups. The C₁₋₇ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₇ alkyls include, without limitation, methyl; ethyl; n-propyl; isopropyl; cyclopropyl; cyclopropylmethyl; n-butyl; iso-butyl; sec-butyl; tert-butyl; and cyclobutyl.

By “C₂₋₇ alkenyl” is meant a branched or unbranched hydrocarbon group containing one or more double bonds and having from 2 to 7 carbon atoms. A C₂₋₇ alkenyl may optionally include a cyclic ring, in which each ring desirably has from three to six members. The C₂₋₇ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl; allyl; 2-cyclopropyl-1-ethenyl; 1-propenyl; 1-butenyl; 2-butenyl; 3-butenyl; 2-methyl-1-propenyl; and 2-methyl-2-propenyl.

By “C₂₋₇ alkynyl” is meant a branched or unbranched hydrocarbon group containing one or more triple bonds and having from 2 to 7 carbon atoms. The C₂₋₇ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₇ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” is meant bromine, chlorine, iodine, or fluorine. By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl,

C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

Other features and advantages of the invention will be apparent from the following Detailed Description, the drawings, and the claims.

### BRIEF DESCRIPTION OF THE DRAWINGS

FIG. 1 is a graph showing the serum concentration of 1-octanol as a function of oral dosage levels (see Example 1). 1-octanol concentrations remained at very low basal levels until the 64 mg/kg dose.

FIG. 2 is a graph showing the serum concentration of octanoic acid (OA) as a function of oral dosage levels of 1-octanol (see Example 1). OA concentrations exhibit a dose-dependency.

FIG. 3 is a graph showing the OA plasma half-life. OA levels were detectable as early as 5-minutes post-dose and levels persisted up to 6 hours following administration (see Example 1). The OA plasma half-life was approximately 74-minutes.

FIG. 4 is a graph showing a peak tremor reduction from baseline of 42% at 120 minutes following 1-octanol dosing, with effects persisting past our 360-minute timepoint (see Example 1). These results suggest that the main effect of oral 1-octanol is mediated via rapid metabolism to OA.

FIG. 5 is a graph showing the effect of octanoic acid on tremor suppression in a harmaline mouse model. Both saline alone and PEG alone failed to suppress harmaline-induced tremor. In contrast, octanoic acid suppressed harmaline-induced tremor in a dose dependent fashion. These results show that octanoic acid, a metabolite of 1-octanol, is useful for the treatment of Essential Tremor.

### DETAILED DESCRIPTION

The invention provides methods, kits, and compositions for treating involuntary tremor by administering octanoic acid or a salt or ester thereof

Applicants have discovered that when 1-octanol is used in the treatment of essential tremor, the plasma levels of 1-octanol are surprisingly low at times and dose levels in which efficacy in reducing tremor is observed. Applicants have correlated this efficacy to circulating plasma levels of octanoic acid, a metabolite formed from 1-octanol. Applicants have also demonstrated, using an animal model for essential tremor, that octanoic acid itself is effective for the treatment of tremor.

Treating involuntary tremor using octanoic acid, or a salt thereof, offers several advantages over therapy using 1-octanol. First, the use of 1-octanol for the treatment of essential tremor is associated with a taste certain patients regard as unpleasant, an unpleasant odor with, and a lingering taste and odor in the mouth post administration. This problem arises in part due to the poor aqueous solubility and lipophilicity of 1-octanol, which floats on the surface of the stomach contents following oral administration. In contrast, octanoic acid and salts of octanoic acid can have reduced volatility and increased aqueous solubility, and therefore are expected to produce fewer side effects, such as unpleasant taste and unpleasant odor. Second, the physical properties of 1-octanol make it difficult to formulate. For example, as a liquid 1-octanol is not easily tabletted, and due to its lipophilicity has a tendency to degrade capsules. Furthermore, 1-octanol is difficult to formulate for injection due to its poor solubility in aqueous solutions. In contrast, an octanoic acid salt can be a solid which is easily formulated in unit dosage form (i.e., either as a capsule or tablet). Because octanoic acid, and salts thereof, can exhibit greater solubility in aqueous solution, they are more easily formulated for injection than 1-octanol. Finally, administering octanoic acid, or a salt thereof, can reduce the variability in the PK profile and response profile (i.e., efficacy) among patients in comparison to 1-octanol therapy as no metabolism of octanoic acid or octanoate is required to produce a therapeutic result. In contrast, it appears that 1-octanol must be first converted to octanoic acid to produce a reduction in involuntary tremor. Because the rate of conversion can vary on a patient by patient basis, it is expected that the response to 1-octanol therapy will be more variable than the therapy described herein.

Pharmaceutical Compositions

The pharmaceutical compositions of the invention include octanoic acid or a salt or ester thereof and a pharmaceutically acceptable excipient. The pharmaceutical compositions can be formulated for oral, topical (including buccal and sublingual), rectal, nasal, vaginal, or parenteral (including subcutaneous, intramuscular, subcutaneous, intravenous, intradermal, intraocular, intratracheal, or intracisternal, intraperitoneal, and epidural) administration.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

Formulations can be used as the active ingredient in combination with one or more pharmaceutically acceptable carrier mediums and/or excipients. As used herein, “pharmaceutically acceptable carrier” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Diluents that can be included in the pharmaceutical compositions of the invention include, without limitation, oils, such as Myverol 18-92, acetylated monoglycerides, Alkamuls 719, Alkamuls 620, Miglyol 812 (caprylic/capric triglyceride), canola oil, caprylic/capric triglyceride, cassia oil, castor oil, castor oil hydrogenated, palm oil-hydrogenated soybean oil, Captex 335 (C8/C10 triglycerides from coconut oil), corn glycerides, corn oil, corn oil PEG-6 esters, cottonseed oil, Captex 200 (C8/ClO diesters of propylene glycol of coconut oil), diacetylated monoglycerides, Sesame oil, Soybean oil hydrogenated, Capmul MCM (C8/C10 mono-/diglycericles from coconut oil), Benzyl Benzoate, Soybean oil, olive oil, PEG vegetable oil, Vegetable oil, Vegetable oil hydrogenated, peanut oil, mineral oil, or vegetable shortening and mixtures thereof.

The flavoring may be one or more flavoring oils. For the purpose of this invention, flavoring oils used herein refer to both entire essential oils and the aroma chemicals malting up the essential oils. Essential oils are predominately volatile materials from botanical sources. The most widely used process for the isolation of essential oils is steam distillation of plant matter, although dry distillation and solvent extraction are also used. Essential oils are generally recognized as safe compositions that can be included in ingested materials. Aroma chemicals refer to chemicals which may be synthetic or natural, derived from essential oils, i.e., derived from plants by distillation, expression, or extraction, and which usually carry the flavor of the plant from which they are derived.

Although the invention is not limited to the specific essential oils listed individually in this specification, a number of important essential oils include: almond-bitter oil, anise oil, anise star dark oil, gurjun balsam oil, white gurjun balsam, basil oil, bergamot oil, camphor oil, caraway oil, cassia oil, cananga oil, chamomile oil, cherry oil, cinnamon oil, citronella oil, clove stern oil, clove leaf oil, clove bud oil, cognac oil, coriander oil, cubeb oil, eucalyptus oil, eugenol oil, ginger oil, grapefruit oil, jasmine oil, laurel oil, lavender oil, lemon oil, lime oil, mace oil, mandarin oil, mayonara oil, menthol oil, mint oil, nutmeg oil, orange oil, patchouli oil, peppermint yakima oil, peppermint oil, rose oil, sage oil, sassafras oil, spearmint oil, tangerine oil, thyme oil, violet oil, vetiver oil, or wintergreen oil.

Aroma chemicals include but are not limited to anethole, carvone, cintronellal, and camphor.

The flavoring may be one or more natural essences, for example an essence derived from Coffee, Tea, Chamomile, Cocoa, Ginger, Grape, Hazelnut, or Guava.

The flavoring may be one or more natural extracts. For example, the flavouring may be almond extract, anise extract, caraway extract, cardamom extract, celery seed extract, chocolate extract, cinnamon extract, clove extract, coriander extract, dark cocoa extract, grand marnier extract, lemon extract, lemon lime extract, lime extract, mandarin mint extract, orange blossom extract, orange extract, parsley herb extract, rum extract, tangerine extract, tarragon extract, or vanilla extract bourbon.

The flavoring may be one or more artificial flavorings, or natural flavorings, such as a spicy flavor (e.g., cinnamon, clove, jalapeno pepper, mace, or nutmeg); a nutty flavor (e.g., almond, butter pecan, cashew, coconut, English walnut-black, hazelnut, peanut, pecan, pistachio, walnut, and walnut-black); commonly used pharmaceutical flavorings having long lasting taste profiles and well characterised taste masking properties (e.g., anise, apple, apricot, banana, blackberry, blueberry, brandy, butter, butter rum, butterscotch, caramel, champagne, cherry-black, cherry-maraschino, cherry-red, cherry-wild, cherry apricot, cherry mint, coconut, coffee, cognac, cola, cranberry, cream soda, currant-black, egg nog, fennel, ginger ale, grape, grapefruit, grenadine, hazelnut, lemon, lemon-lime, maple, maple walnut, mint orange, passion fruit, peach, pineapple, plum, prune, raspberry, root beer, rum, rum & coffee, sherry, spearmint, tangerine, tutti frutti, or vanilla custard).

Flavorings such as aroma chemicals, natural essences, essential oils, natural extracts, artificial flavors, natural flavors and pharmaceutical flavors are commonly available, for example, from Blue Pacific Flavours & Fragrances, Inc., (1354 South Marion Court, City of Industry, Calif., USA 91745-2418).

Additionally, the formulations can be combined with pharmaceutically acceptable excipients, and, optionally, sustained-release matrices, such as biodegradable polymers, to form therapeutic formulations. A “pharmaceutically acceptable excipient” includes a non-toxic solid, semi-solid or liquid filler, diluent, encapsulating material or formulation auxiliary of any type.

Capsules/tablets

Pharmaceutical compositions formulated for oral administration will generally include an inert diluent or edible carrier. These can be prepared in unit dosage forms such as, but not limited to, capsules, gel capsules, tablets, caplets, pills, troches or cachets, each containing a predetermined amount of octanoic acid, or a salt or ester thereof. The unit dosage formulation can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include but are not limited to microcrystalline cellulose, gum tragacanth, glucose solution, acacia mucilage, gelatin solution, molasses, polvinylpyrrolidine, povidone, crospovidones, sucrose and starch paste. Lubricants include but are not limited to talc, starch, magnesium or calcium stearate, Sterotes, lycopodium and stearic acid. Diluents include but are not limited to, for example, lactose, sucrose, starch, kaolin, salt, mannitol and dicalcium phosphate. Glidants include, but are not limited to, colloidal silicon dioxide. Disintegrating agents include but are not limited to crosscarmellose sodium, sodium starch glycolate, alginic acid, Primogel, corn starch, potato starch, bentonitc, methylcellulose, agar and carboxymethylcellulose. Coloring agents include but are not limited to, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include but are not limited to natural flavors extracted from plants such as fruits and synthetic blends of compounds which produce a pleasant sensation, such as, but not limited to peppermint and methyl salicylate. Wetting agents include but are not limited to propylene glycol monostearate, sorbitan monooleate, diethylene glycol monolaurate and polyoxyethylene laural ether. Emetic-coatings include but are not limited to fatty acids, fats, waxes, shellac, ammoniated shellac and cellulose acetate phthalates. Film coatings include but are not limited to hydroxyethylcellulose, sodium carboxymethylcellulose, polyethylene glycol 4000 and cellulose acetate phthalate.

When the unit dosage form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. Encapsulating substances for the preparation of enteric-coated oral formulations include cellulose acetate phthalate, polyvinyl acetate phthalate, hydroxypropyl methylcellulose phthalate and methacrylic acid ester copolymers.

Syrups/Elixirs

The pharmaceutical compositions of the invention can be formulated as a syrup, elixir or drink suitable for oral administration. Liquid formulations can, for example, be prepared by dissolving, dispersing, or otherwise mixing the octanoic acid, or a salt or ester thereof, in a carrier, such as a solid or liquid filler, diluents, to thereby form a syrup, elixir, solution or suspension. A syrup can contain, in addition to the active compounds, sucrose as a sweetening agent and certain preservatives, dyes and colorings and flavors. Such liquid preparations can be prepared by conventional means with pharmaceutically acceptable additives such as suspending agents, such as sorbitol syrup, cellulose derivatives or hydrogenated edible fats; emulsifying agents such as lecithin or acacia; non-aqueous vehicles such as soybean oil, almond oil, oily esters, ethyl alcohol or fractionated vegetable oils; preservatives such as methyl or propyl-p-hydroxybenzoates or sorbic acid, buffer salts, flavoring agents, coloring agents and sweetening agents as appropriate. Methods of preparing such dosage forms are known, or will be apparent, to those skilled in this art; for example, see Remington's Pharmaceutical Sciences, Mack Publishing Company, Easton, Pa., 15th Edition, 1975.

Parenteral Formulations

Alternatively, the pharmaceutical compositions of the invention can be formulated for parenteral, intradermal, subcutaneous, or topical application. Such formulations can include the following components: a sterile diluent such as water for injection, saline solution, aqueous and non-aqueous sterile suspensions which can include suspending agents and thickening agents, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

If administered intravenously, preferred carriers are physiological saline or phosphate buffered saline (PBS). The formulations can be presented in unit-dose or multi-dose containers, for example, sealed ampoules, disposable syringes and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules, and tablets of the kind previously described above.

Pharmaceutical organic or inorganic solid or liquid carrier media suitable for enteral or parenteral administration can be used to fabricate the formulations. Gelatin, lactose, starch, magnesium stearate, talc, vegetable and animal fats and oils, gum, polyalkylene glycol, water, or other known carriers can all be suitable as carrier media.

Buccal/Lingual Spray

The pharmaceutical compositions of the invention can be formulated for buccal or sublingual administration. Formulations suitable for buccal or sublingual administration include for example, tablets or lozenges, having the ingredients in a flavored basis, usually sucrose and acacia or tragacanth; pastilles, having one or more of the formulations disclosed herein in an inert basis such as gelatin and glycerin, or sucrose and acacia and lingual sprays and mouthwashes, having one or more of the formulations disclosed herein administered in a suitable liquid carrier. The components of aerosol formulations include solubilized active ingredients, antioxidants, solvent blends and propellants for solution formulations, and micronized and suspended active ingredients, dispersing agents and propellants for suspension formulations.

Nasal

The pharmaceutical compositions of the invention can be formulated for nasal or intranasal administration. Formulations suitable for nasal administration, when the carrier is a solid, include a coarse powder having a particle size, for example, in the range of approximately 20 to 500 microns which is administered by rapid inhalation through the nasal passage. When the carrier is a liquid, for example, a nasal spray or as nasal drops, one or more of the formulations can be admixed in an aqueous or oily solution, and inhaled or sprayed into the nasal passage. For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount, Capsules and cartridges of, for example, gelatin for use in an inhaler or insufflator can be formulated containing a powder mix of the compound and a suitable powder base such as lactose or starch.

Topical

The pharmaceutical compositions of the invention can be formulated for topical administration. Formulations suitable for topical administration to the skin can be presented as ointments, creams, gels, and pastes, having one or more of the formulations administered in a pharmaceutical acceptable carrier. The formulations can contain additional agents, such as cleaning agents, wetting agents, sequestering agents, tonicity agents, nutrient agents, contact lens conditioning agents, antioxidants, pH adjustors, and the like. These additional components can be included in the present formulations in an amount effective to impart or provide the beneficial or desired property to the formulations.

Rectal

The pharmaceutical compositions of the invention can be formulated for rectal administration. Formulations for rectal administration can be presented as a suppository with a suitable base containing, for example, cocoa butter or a salicylate.

Vaginal

The pharmaceutical compositions of the invention can be formulated for vaginal administration. Formulations suitable for vaginal administration can be presented as pessaries, tampons, creams, gels, pastes, foams or spray formulations containing one or more of the formulations and appropriate carriers.

Controlled or Sustained Release

The pharmaceutical compositions of the invention can be formulated for controlled release, delayed release, or combined delayed and controlled release profile. Alternatively, the formulation can present a combination of an immediate release formulation and a controlled release formulation.

The controlled-release of octanoic acid, or a salt or ester thereof, can be controlled in any way suitable for achieving the desired result. Books describing methods of controlled delivery that are appropriate for the delivery of octanoic acid include: Robert S. Langer, Donald L. Wise, editors; Medical applications of controlled release (Volumes 1 and 2); Boca Raton, Fla.: CRC Press, 1984; and William J. M. Hrushesky, Robert Langer and Felix Theeuwes, editors; Temporal control of drug delivery (series); New York: New York Academy of Sciences, 1991. Representative, non-limiting systems encompassed by the present invention include diffusion-controlled, solvent-controlled and chemically-controlled systems.

For example, a controlled-release of octanoic acid, or a salt or ester thereof, can be achieved using a diffusion-controlled system, which can be for example, a reservoir device, such as a membranes, capsules, microcapsules, liposomes, or hollow fiber; or a monolithic (matrix) device, such as a polymer matrix. In a particular embodiment of the invention, a polymeric film coating is an enteric polymeric film coating allows the coated solid to pass intact through the stomach to the small intestine, where the drug can then be released for absorption through the intestinal mucosa into the human body where it can exert its pharmacologic effects. Non-limiting examples of enteric polymers include cellulose, vinyl, and acrylic derivatives. In another embodiment, sustained release of octanoic acid can be achieved through microencapsulation. The microencapsulation drug delivery system of the present invention can utilize a variety of protective wall or covering materials, including without limitation, proteins, polysaccharides, starches, waxes, fats, polymers and resins. Polymers can be natural, synthetic or synthetically modified natural polymers. Representative, non-limiting polymers include gelatins, fish collagens, rubber arrabicum, silicon rubber albumen, fibrinogens, casein, haemoglobin, zein, alginate, nylon, nylon-polyethylenimine carragheen, agar-agar, chitosan, arabino-galactan, gelan, cellulose, polyvinylalcohol, polyacroleins, polylactic acid, polyglycolic acid polyamides, polyethyleneglycols, ethyl Styrolmaleinacidanhydride copolymers, cellulosesulphate-poly(dimethyldiallyl)-ammonium chloride, hydroxy-ethyl methacrylate-methyl methacrylate, chitosan-carboxymethyl-cellulose and alginate-polylysine-alginate.

Polymers suitable for use in the formation of monolithic matrix devices include naturally occurring polymers, synthetic polymers and synthetically modified natural polymers. The monolithic matrix device of the present device can also contain polymer derivatives. As used herein, “derivatives” include polymers having substitutions, additions of chemical groups, for example, alkyl, alkylene, hydroxylations, oxidations, and other modifications routinely made by those skilled in the art.

Any polymeric plastic material is suitable for use in the present invention provided it is insoluble or substantially insoluble in water, and includes cellulose derivatives such as cellulose acetates, (cellulose acetate butyrate, cellulose acetate propionate, cellulose acetate phthalate, etc.), methyl, ethyl and propyl celluloses; polycarbonates; polystyrenes; alkylacrylates such as polymethyl methacrylate, polyethyl ethacrylate, polyethylene, polyethylene methacrylate and other lower alkyl acrylates; vinyl acetate/vinyl chloride, methyl acrylate/methylmethacrylate vinyl polymers; polyvinylchloride polyurethanes; polyacrylonitriles; and mixtures, combinations and multipolymers (copolymers, terpolymers, etc.) thereof.

Solvent activated systems include (i) swellable controlled-release systems, such as a hydrogel; (ii) osmotic systems (i.e., involving transport of water through a semipermeable membrane).

For example, the polymer matrix can be a hyroxypropylmethylcellulose (HPMC) matrix. (See generally Hogan J E. “Hydroxypropylmethylcelluose sustained release technology, Drug Dev. Ind. Pharm. (1989) 15, 975-999). The HPMC matrix can include an HPMC homopolymer, co-polymer or terpolymer. A single HPMC or a mixture of HPMCs of difference molecular weight and structure can be used. HPMC can be used alone, or in a combination with a second polymer type to form a polymer blend.

Osmotically controlled systems are also suitable for use in the present invention. In this embodiment, an osmotic pressure gradient is created to draw an aqueous fluid into a compartment containing octanoic acid, causing octanoic acid to be delivered. Osmotic delivery systems include a compartment containing octanoic acid and an osmotic agent which thaws an aqueous fluid through the walls of the compartment, causing swelling of the osmotic agent and delivery of octanoic acid.

Alternatively, the drug delivery system is a chemically controlled system. Chemical control can be achieved, for example, using bioerodable polymers or pendant chains. For example, controlled release of octanoic acid can be achieved using a biodegradable monolithic polymer matrix. In this type of system, the bioactive agent is ideally distributed uniformly throughout a polymer in the same way as in monolithic systems. Biologically degradable polymers are polymers which degrade to smaller fragments due to chemicals present inside the body. In generally, biologically degradable polymers are either (i) biodegradable polymers or (ii) bioabsorbable polymers. Biodegradable polymers degrade to smaller fragments by enzymes, whereas bioabsorbable polymers degrade in the presence of other chemicals in the body. Biodegradable polymers include (i) naturally occurring polymers; (ii) modified natural polymers (i.e., chemically or enzymatically modified polymers; and (iii) synthetic polymers. Representative, non-limiting, naturally occurring biodegradable polymers include alginate, dextrin, cellulose, collagen, chitosan and proteins such as albumin, zein and copolymers and blends thereof, alone or in combination with synthetic polymers. In general, these materials degrade either by enzymatic hydrolysis or exposure to water in vivo, by surface or bulk erosion.

Geometrical-physical systems can be used to provide controlled-release octanoic acid. This type of system incorporates octanoic acid, or a salt or ester thereof, into a layer a layer or core, which is then formed into a pellet and altered by physical means to effect and control the rate or erosion or dissolution of the dosage form. Surface-area modifications are used to retard the burst release or increase the extent of the release of octanoic acid from tablet cores that possess diffusion limitations. The physically-altered pellet can then be incorporated alone or in combination with other modified pellets and excipients into a capsule or tablet. Representative geometrical-physical systems include enteric-coated tablet, modified-core tablet systems (e.g., Procise®, GlaxoSmithKline; Smartrix®, Smartrix Technologies).

Non-limiting examples of other polymers suitable for use in the controlled-release drug delivery system according to the present invention include gelatins, fish collagens, rubber arrabicum, silicon rubber albumen, fibrinogens, casein, haemoglobin, zein, alginate, nylon, nylon-polyethylenimine carragheen, agar-agar, chitosan, arabino-galactan, gelan, cellulose, polyvinylalcohol, polyacroleins, polylactic acid, polyglycolic acid polyamides, polyethyleneglycols, ethyl Styrolmaleinacidanhydride copolymers, cellulosesulphate-poly(dimethyldiallyl)-ammonium chloride, hydroxy-ethyl methacrylate-methyl methacrylate, chitosan-carboxymethyl-cellulose, alginate-polylysine-alginate, cellulose ester, cellulose ether, an acrylic polymer, ethyl cellulose, cellulose acetate, cellulose acetate butyrate, poly(lactide-co-glycolide) (PLGA), poly(lactic acid) (PLA), poly(glycolic acid) (PGA), polyvinyl chloride, polyethylene, vinyl acetate/vinyl chloride copolymers, polymethylmethacrylates, polyamides, silicones, polystyrene low density polyethylene, ethylene-vinylacetate copolymers, styrene-butadiene-styrene copolymers, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), hydro glycerides (e.g., mono-, di- or triglycerides such as stearin, palnitin, laurin, myristin, hydrogenated castor or cottonseed oils, precirol), fatty acids and alcohols (e.g., stearic, palmitic or lauric acids; stearyl, cetyl or cetostearyl alcohols), fatty acid esters (e.g., monostearates of propylene glycol and of sucrose, sucrose distearate), waxes (e.g., white wax, cachalot wax), hydrogenated castor oil (HCO), ethylcellulose, poly(hydroxy acids), poly(lactic acid), poly(glycolic acid), poly(lactic acid-co-glycolic acid), poly(lactide), poly(glycolide), poly(lactide-co-glycolide), polyanhydrides, polyorthoesters, polyamides, polycarbonates, polyalkylenes, polyethylene and polypropylene, polyalkylene glycols, poly(ethylene glycol), polyalkylene oxides, poly(ethylene oxide), polyalkylene terepthalates, poly(ethylene terephthalate), polyvinyl alcohols, polyvinyl ethers, polyvinyl esters, poly (dimethyl silicone) polymethacrylate, polymethylmethacrylate, polyvinyl halides, poly(vinyl chloride), polyvinylpyrrolidone, polysiloxanes, poly(vinyl alcohols), poly(vinyl acetate), poly (ethylene/vinyl acetate)polystyrene, polyurethanes, derivativized celluloses, alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, polymers of acrylic acid, methacrylic acid, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(butyric acid), poly(valeric acid), poly(lactide-co-caprolactone), polyphosphazenes, poly(vinyl alcohols), polyamides, polycarbonates. polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, polyacrylates, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate), albumin, prolamines, cellulose, dextrans, polyhyaluronic acid, polyhydroxyalkanoates, polyhydroxybutyrate, alkyl celluloses, hydroxyalkyl celluloses, nitrocelluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, ellulose sulfate sodium salt, ethylcellulose, polyvinyl alcohol, hydroxypropylmethylcellulose, olymethylmethylacrylate, ethyl acrylate, polyethylene, polyvinylacetate, polymethacrylate, styrene/maleic copolymer, cellulose acetate pthalate, cellulose acetate pthalate/PEG blend, microcrystalline cellulose, polydextrose, lactose, shellacs, cellulose derivatives, non-cellulose polysaccharides, polyethylene oxide, polyvinyl alcohols, acrylic acid copolymers methylcellulose, hydroxypropyl methylcellulose (IHPMC) (high, medium and low molecular weight), hydroxyethyl cellulose, hydroxypropyl cellulose, carboxymethylcellulose, hydroxomethylcellulose, hemicellulose, methylcellulose, galactomannans, guar gum, carob gum, gum arabic, sterculia gum, agar, alginates, carbopols 934P and 974P, polyvinyl alcohol (PVA)/polyvinyl pyrrolidone (PVP), gum tragacanth, locust bean gum, karaya gum, proteinaceous substances (e.g., pectin, carrageen) carboxypolymethylene, gelatin, bentonite, magnesium aluminum silicate, carbomer, zooglan, polysaccharides, modified starch derivatives (e.g., Amazio 721A), hydrophilic vinyl acrylic polymers, poly(2-hydroxyethyl methacrylate), poly(acrylic acid), poly(methacrylic acid), poly(N-vinyl-2-pyrohdinone), poly(vinyl alcohol) polyanhydrides, polyesters, polyacrylic acids polyurethanes, pollyphosphoesters and polyphosphazenes and poly(methyl methacrylates, polyanhydrides, poly(ortho)esters, polyurethanes, poly(butyric acid), poly(valeric acid), poly(lactide-co-caprolactone), poly(ethylene terephthalate), poly(butyric acid), poly(valeric acid), poly(lactide-co-caprolactone), polyanhydrides, starch-polyester alloys; styrene-maleic anhydride copolymers, poly(methylvinyl ether-maleic acid), starch, starch-PCL blends, polylactic acid (PLA)-starch blends, polylactic acid, poly(lactic acid-glycolic acid) copolymers, polylactide, polyglycolide; polyactide co-glycolide PC, starch esters, starch ester-aliphatic polyester blends, modified corn starch, polycaprolactone, poly(n-amylmethacrylate), ethyl cellulose, wood rosin, polyvinylalcohol (PVOH), polyhydroxybutyrate-valerate (PHBV), biodegradable aliphatic polyesters, polyhydroxybutyrate (PHB) and polyhydroxy acids.

The controlled-release formulation can also include a number of other excipients and diluents. The tern “excipient' refers to substances that are commonly provided within finished dosage forms, and include vehicles, binders, disintegrants, fillers (diluents), lubricants, glidants (flow enhancers), compression aids, colors, flavors sweeteners, preservatives, suspending/dispersing agents, film formers/coatings and printing inks.

Lubricants can include, for example, magnesium stearate, calcium stearate, zinc stearate, powdered stearic acid, hydrogenated vegetable oils, talc, polyethylene glycol, and mineral oil;

Disintegrants include starches such as corn starch, potato starch, pregelatinized and modified starches thereof cellulosic agents such as Ac-di-sol, montmorrilonite clays, cross-linked PVP, sweeteners, bentonite and VEEGUM™, microcrystalline cellulose, alginates, sodium starch glycolate, gums such as agar, guar, locust bean, karaya, pectin and tragacanth. The present formulations can also contain flavorants or sweetening agents.

The octanoic acid-containing pharmaceutical formulation can require particular binders in order to obtain a suitable control-release product. Suitable binders include but are not limited to cellulose acetate butyrate, cellulose acetate propionate, cellulose propionate high molecular weight (200,000), cellulose propionate medium molecular weight (75,000), cellulose propionate low molecular weight (25,000), cellulose acetate, cellulose nitrate, ethylcellulose, polyvinyl acetate, polyvinylpyrrolidone, vinyl alcohol polymer, polyethylene oxide, water soluble or water swellable cellulose and starch derivatives, acacia, tragacanth, gelatin, starch, cellulose materials such as methyl cellulose and sodium carboxymethyl cellulose, alginic acids and salts thereof, polyethylene glycol, guar gum, polysaccharide, sugars (e.g., lactose, sucrose), invert sugars, poloxomers (PLURONIC™ F68, PLURONIC™ F127), collagen, albumin, gelatin, cellulosics in nonaqueous solvents, pregelatinized starch, starch paste and combinations of the above and the like. Other binders include, for example, polypropylene glycol, polyoxyethylene-polypropylene copolymer, polyethylene ester, polyethylene glycol, polyethylene sorbitan ester, polyethylene oxide or combinations thereof and others known to those of ordinary skill in the art.

The pharmaceutical formulations can also contain diluents. The term diluent is intended to mean inert substances used as fillers to create the desired bulk, flow properties, and compression characteristics in the preparation of tablets and capsules. Such compounds include, by way of example and without limitation, dibasic calcium phosphate, kaolin clay, fructose, sucrose, dextrose, lactose, mannitol, microcrystalline cellulose, powdered cellulose, precipitated calcium carbonate, sorbitol, calcium sulfate, starch and the like. The formulations can contain colorants.

Dosing Regimens

The pharmaceutical compositions of the invention can contain 1-2000 mg of octanoic acid, or a salt or ester thereof (e.g., at least about 1, 5, 10, 100, 200, 300, 400, 500, 600, 700, 800, 900, 1000, 1500, 2000, or 5000 mg of octanoic acid, or a salt or ester thereof).

The dose of octanoic acid, or a salt or ester thereof, for the treatment of tremors can be in the range from about 1 to 50 mg/kg, preferably 1 to 20 mg/kg, of body weight per day, more generally 0.1 to about 100 mg per kilogram body weight of the recipient per day. Lower doses can be, for example, doses of 0.5-100 mg, 0.5-50 mg, 0.5-10 mg, or 0.5-5 mg per kilogram body weight per day.

The octanoic acid, or a salt or ester thereof, is conveniently administered in unit any suitable dosage form, including but not limited to one containing 1 to 3000 mg, preferably 70 to 1400 mg of active ingredient per unit dosage form. An oral dosage of 50-1000 mg is usually convenient, including in one or multiple dosage forms of 50, 100, 200, 250, 300, 400, 500, 600, 700, 800, 900 or 1000 mgs. Lower doses can be preferable, for example from 10-100 or 1-50 mg. Also contemplated are doses of 0.1-50 mg, or 0.1-20 mg or 0.1-10.0 mg. Furthermore, lower doses can be utilized in the case of administration by a non-oral route, as, for example, by injection or inhalation.

The concentration of octanoic acid, or a salt or ester thereof, will depend on absorption, inactivation, and excretion rates of the drug as well as other factors, such as the disorder being treated, severity of the disorder, the specific formulation employed, the age, body weight, general health, sex and diet of the patient, the time of administration, route of administration, and the duration of the treatment. It is to be understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The octanoic acid, or a salt or ester thereof, can be administered at once, or can be divided into a number of smaller doses to be administered (e.g., parenterally, transmucosally, transdermally, intramuscularly, intravenously, intradermally, subcutaneously, intraperitonealy, intraventricularly, or intracranially) at varying intervals of time during a day. The dosage administered can be, for example, in the range of approximately 1-750 mg/day (e.g., 5-80 mg/day, 35-66 mg/day, 40-60 mg/day, 45-60 mg/day, 15-25 mg/day, 55-65 mg/day, 45-60 mg/day, 60 mg/day, 20 mg/day, or 45 mg/day).

Therapy

The methods and compositions of the invention can be used for the treatment of involuntary tremors, such as essential tremor, drug-induced tremor, or disease-induced tremor. Subjects who can benefit from the methods and compositions of the invention include those suffering from, for example Parkinson's disease, multiple sclerosis, stroke, head injury Wilson's disease, mercury poisoning; an over-active thyroid gland; liver encephalopathy, or tremor associated with psychiatric disorders.

Involuntary tremor that can be treated with the compositions and methods described herein include, but are not limited to tremor associated with the following: normal aging, stress, tiredness, caffeine, alcohol, hypoglycemic attack, high blood sugar levels, anger, aggression, excitement, alcoholism, liver disease, kidney disease, stroke, hypoglycemia, brain tumors, hyperthyroidism, Friedrich's ataxia, head injury, concussion, tertiary syphilis, syphilis, seizure disorders, anxiety disorders, GAD, panic disorders, intermittent explosive disorder, alcohol withdrawal, drug withdrawal, amphetamine withdrawal, hallucinogen withdrawal, illicit drugs, cocaine, amphetamine intoxication, asthma medications, theophylline, epileptic medications, dilantin, compazine,

African Sleeping sickness, autoimmume thyroid diseases, barbiturate abuse, benzodiazepine abuse, bipolar disorder, carbon monoxide poisoning, delirium tremens, dementia with lewy bodies, diabetic hypoglycemia, dystonias, Epilepsy, Fahr's Syndrome, Febrile Seizures, Friedreich's ataxia, Generalized anxiety disorder, Graves Disease, hyperthyroidism, hypoglycemia, Japanese encephalitis, Machado-Joseph Disease, Malaria, metachromatic Leukodystropliy, Multiple Sclerosis, Olivopontocerebellar atrophy, Opsoclonus Myoclonus, Panic attack, Pelizaeus-Merzbacher disease, rabies, Ramsay Runt Syndrome Type 2, Schilder's Disease, social phobia, Spinal Muscular Atrophy type III, Temporal arteritis, thyroid disorders, Toxoplasmosis and/or Wilson's

Disease.

The octanoic acid, or a salt or ester thereof, can be administered to treat tremor occurring as a side effect of another therapeutic agent. Tremor can occur as a side effect of drugs including neuroleptics, metoclopramide, theophylline, bronchodilators, Valproate, amiodarone, stimulants, such as cocaine and amphetamines; antidepressants, antipsychotics, caffeine, lithium, a variety of medications used to treat Parkinson's disease, asthma medications, thyroid hormone medications, and as a result of withdrawal from alcohol or addictive drugs. Types of tremors induced by drugs include enhanced physiologic tremor, rest tremor, and action tremor. Signs and symptoms of drug-induced tremors depend on the drug used and on a patient's predisposition to its side effects. Some drugs can cause extrapyramidal side effects manifesting as bradykinesia, rigidity, and tremor.

The drug-induced tremor to be treated can be caused by a drug selected from but not limited to the following: cysclosporine, antidepressants, such as selective serotonin reuptake inhibitors (SSRI's) (for example, prozac, paxil);

amphetamines, antipsychotics, such as haloperidol, lithium, and metoclopramide; and/or caffeine. In one embodiment, the drug-induced tremor can be caused by withdrawal from drugs such as alcohol and/or opiods.

Combination Therapy

The octanoic acid, or a salt or ester thereof, can be administered in combination with a second drug useful for the treatment of tremor, such as beta blockers (e.g., propranolol, nadolol and/or metoprolol); anticonvulsants (e.g., primidone, acetazolamide, methazolamide, valproic acid and/or gabapentin); benzodiazepines (e.g., diazepam, alprazolam, chlordiazepoxide, and clonazepam); and Botulinum toxin type A and/or B.

The octanoic acid, or a salt or ester thereof, can be administered in combination with drugs used to treat Parkinson's disease. These include, without limitation, dopaminergic agents, such as L-dopa and/or levodopa/carbidopa, dopamine agonists, such as bromocriptine, pramipexole, ropinirole and/or pergolide; COMT inhibitors, such as entacapone and/or tolcapone; monoamine oxigenase (MAO) inhibitors, such as selegiline and/or rasagiline; antiexcitatory agents, such as remacemide; anti-viral agents, such as amantadine; anticholinergics, such as trihexyphenidyl, benztropine, procyclidine, and/or biperiden; antipsychotics, such as risperidone, olanzapine, quetiapine, and/or clozapine.

The octanoic acid, or a salt or ester thereof, can be administered in combination with drugs used to treat multiple sclerosis. In one embodiment, the octanoic acid formulations described herein can be given in combination and/or alternation with a drug selected from but not limited to the following: interferon beta 1 a, interferon beta 1 b, glatiramer acetate, mitoxantrone, azathiprine, cyclophosphamide, cyclosporine, methotrexate, cladribine, methylprednisolone, prednisone, prednisolone, dexamethasone, acth, corticotrophin, carbamazepine, gabapentin, topiramate, zonisamide, phenytoin, desipramine, amitriptyline, imipramine, doxepin, protriptyline, cannabis, pentoxiffylline, and/or hydroxyzine.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

Therapeutic Effect of 1-Octanol Mediated by Conversion to Octanoic Acid in Adults with Essential Tremor.

Early studies of 1-octanol in humans with Essential Tremor (ET) have shown it to be safe and effective at tremor suppression (see Bushara et al.,

Neurology 62:122 (2004); and Shill et al., Neurology 62:2320 (2004)). Based on this previous work, we conducted a study of 1-octanol to characterize its pharmacokinetic profile.

Methods

Subjects: All subjects underwent a routine medical screening and neurological examination. A structured ethanol challenge was carried out to confirm tremors were ethanol-responsive. Subjects were videotaped at 0, 15, 30, 45, 60, and 75-minutes after receiving two servings (50-ml each) of 40% ethanol along with digitized spirography measures. Ethanol-responsive subjects then completed either a dose titration (1-64 mg/kg) inpatient study or a fixed dose (64 mg/kg) inpatient study to characterize the pharmacokinetics of 1-octanol and its predicted primary metabolite octanoic acid (OA).

Plasma Sampling: During the inpatient study, subjects received oral 1-octanol followed by collection of plasma samples at the following intervals: 5, 20, 45, 70, 100, 130, 160, 210, 270 and 360-minutes post-dose. Samples were processed then batch analyzed using high-performance liquid chromatography and mass spectrometry methods to measure concentrations of 1-octanol and OA.

Tremor Assessment: To assess efficacy, subjects drew Archimedes spirals at fixed intervals on a digitizing tablet with an overlaid spiral guide. We used in-house analysis software to process the data before conducting a fast Fourier transform of tremor velocity to obtain our frequency and power measures.

Results

Four subjects completed the dose titration phase of the study and 10 subjects completed the fixed dose phase.

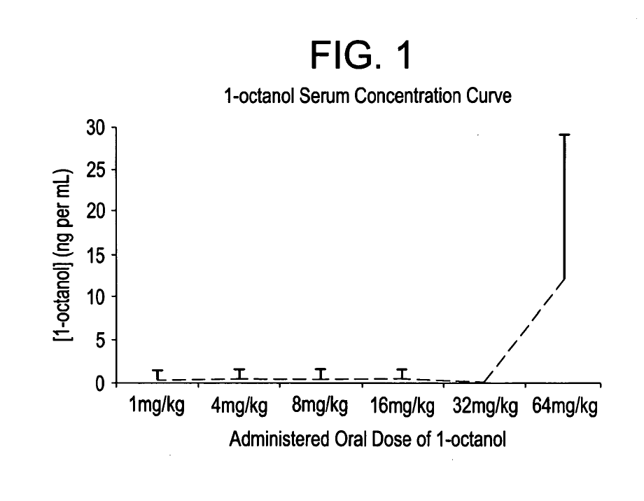

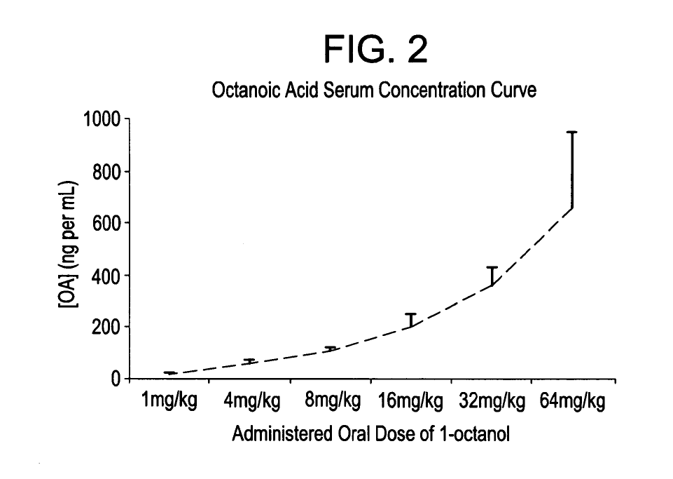

1-octanol concentrations remained at very low basal levels until the 64 mg/kg dose (see FIG. 1), while OA concentrations showed a dose-dependency (see FIG. 2).

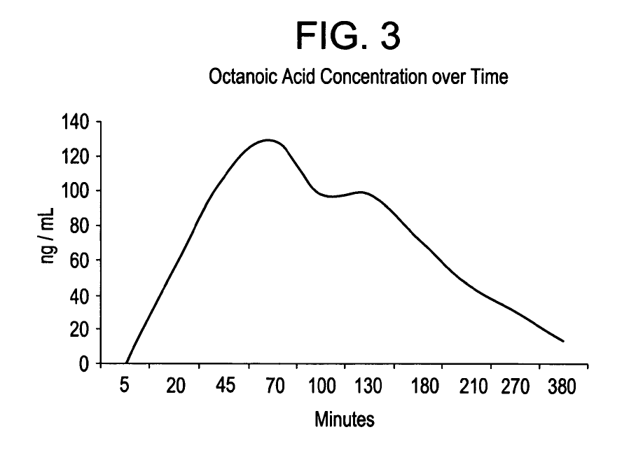

Plasma concentrations of OA were detectable as early as 5-minutes post-dose and levels persisted up to 6 hours following administration (see FIG. 3). The OA plasma half-life was approximately 74-minutes.

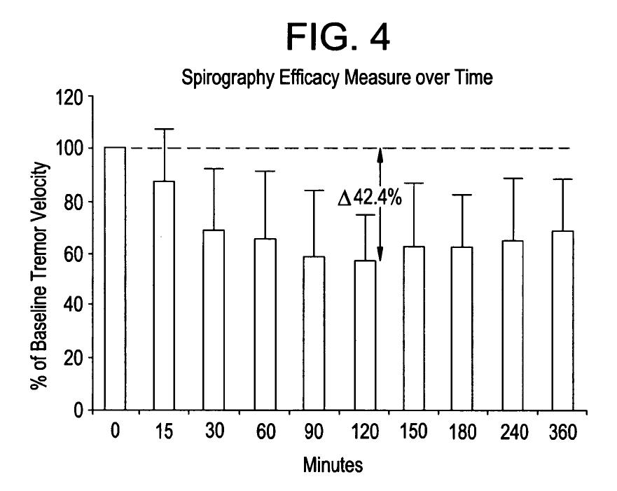

Efficacy measures showed a peak tremor reduction from baseline of 42% at 120 minutes, with effects persisting past our 360-minute timepoint (see FIG. 4).

### Conclusions

Our results suggest that the main effects of oral 1-octanol are mediated via rapid metabolism to OA. The efficacy measures are known to be dependent upon the dosing level of 1-octanol, but here it is shown that these measures do not reflect the circulating concentration of 1-octanol. Rather, the-efficacy measures parallel the circulating concentrations of OA, and not 1-octanol.

### EXAMPLE 2

Pre-clinical Assessment of Octanoic Acid for Anti-tremor Efficacy in the Harmaline Mouse Model of Essential Tremor.

Methods

The harmaline model of essential tremor: Harmaline is a beta-carboline alkaloid that produces action tremor on systemic administration to mammals (see Llinas et al., Exp Brain Res 18:69 (1973)). It induces rhythmic burst-firing of inferior olivary neurons that is associated with Purkinje discharges at the tremor frequency (Bernard et al., Exp Brain Res 57:128 (1984)). In inferior olive slices, harmaline promotes synchronized oscillations (Llinas et al., J Physiol 376:163 (1986)). Tremor in this model is abolished by lesioning the inferior olive (Simantov et al., Brain Res 114:144 (1976)). Harmaline thus acts within the olivo-cerebellar system to produce tremor and can be used as a model to assess potential ET therapies (see Martin et al., Mov. Disord. 20:298 (2005)).

Animals: Male ICR mice (20-24 g) from Harlan (Indianapolis, Ind.) were housed in groups in standard cages with ad libitum access to commercial rodent diet and water.

Tremor measurement: Motion activity was measured with a Convuls-1 Replacement Sensing Platform model 1335-1A (Columbus Instruments, Columbus, Ohio), a metal platform with a load sensor beneath it, that is connected to a Grass model P511 AC amplifier (Grass Instruments, West Warwick, R.I.) with 1 and 70 Hz filter settings. Digitally recorded motion power was analyzed using Cambridge converter and software. Data was sampled at 128 Hz. The total motion power for each 20-minute epoch was calculated between 0 and 34 Hz (full motion spectrum) and between 10 and 16 Hz (the harmaline tremor frequency bandwidth); and the motion power percentage (10-16 Hz power)/(0-34 Hz power)×100 then calculated. This percentage is expressed as motion power percentage (MPP). In untreated normal mice, the MPP is approximately 25-30 percent and represents the proportion of normal motor activity falling within the 10-16 Hz bandwidth rather than tremor.

Tremor measurement protocol: Each mouse was placed within a black plexiglass cage on the tremor platform and allowed 10 minutes for habituation before the collection of 20 minutes of pre-harmaline baseline motion power data. Harmaline, 20 mg/kg s.c. in a volume of 4 ml saline/kg, was then administered, and motion power measurement initiated again 20 minutes later when full tremor had stabilized. Harmaline tremor was then recorded for 20 minutes to assess the adequacy of the tremor response, defined as an MPP increase of at least 20 percent over pre-harmaline baseline. The test drug or vehicle was then administered (this time defined at time 0) and motion power recorded for five successive 20-minute epochs. Harmaline-induced tremor typically lasted 100 minutes. Data from mice which failed to show an adequate tremor response to harmaline was not used. In addition, data from mice whose baseline MPP was outside the 95% confidence interval was not used. This experimental design provides a non-harmaline baseline MPP; if the intervention reduces MPP to this level, complete tremor suppression is inferred. The harmaline pre-drug session was used to show that the groups had comparable tremor prior to the intervention.

Drugs and drug administration: Harmaline HCl was obtained from

Sigma (St. Louis, Mo.). Octanoic acid and polyethylene glycol vehicle (PEG-300) were administered i.p. in a volume of 3.5 ml/kg.

Octanoic acid efficacy: Mice were assigned to the following groups: (1) saline alone, (2) PEG alone, (3) PEG plus octanoic acid at partly effective tremor-suppressive dose, and (4) PEG plus octanoic acid at completely effective dose. There were 6 to 10 mice in each group to power an adequate comparison of the groups. A repeated measures ANOVA model were applied to MPP values in each experiment followed by post-hoc t-tests under the model using the Tukey-Fisher significance criterion. The descriptive statistic percent change in tremor were calculated for the first and second treatment epochs with the assumption that random non-tremor motion power within the tremor bandwidth is unchanged during treatment compared to pre-harmaline baseline.

Results

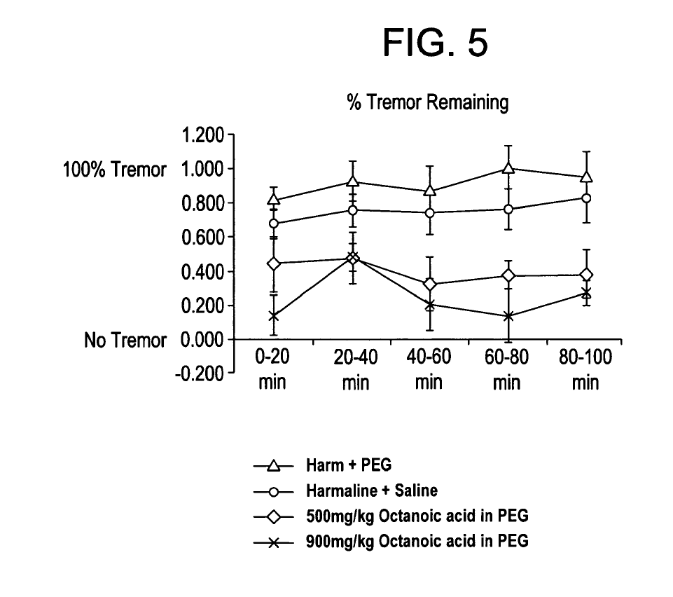

The effect of octanoic acid on tremor suppression is shown in FIG. 5.

Both saline alone and PEG alone failed to suppress harmaline-induced tremor. In contrast, octanoic acid suppressed harmaline-induced tremor in a dose dependent fashion.

Conclusions

Our results show that octanoic acid, a metabolite of 1-octanol, is useful for the treatment of Essential Tremor.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A pharmaceutical composition in unit dosage form comprising octanoic acid, or a salt or ester thereof, in an amount sufficient to treat involuntary tremors when administered to a subject.

2. The pharmaceutical composition of claim 1, wherein said unit dosage form comprises from 1 mg to 1.0 g of octanoic acid, or a salt or ester thereof.

3. The pharmaceutical composition of claim 2, wherein said unit dosage form comprises octanoic acid.

4. The pharmaceutical composition of claim 2, wherein said unit dosage form comprises an ester of octanoic acid.

5. The pharmaceutical composition of claim 4, wherein said ester is 1-methyl-1,2-ethanediyl octanoate (1,2-dicaprylin), methyl octanoate, ethyl octanoate, propyl octanoate, butyl octanoate, hexyl octanoate, heptyl octanoate, octyl octanoate, octanoylglucuronide, or 1,2,3-propanetriyl octanoate.

6. The pharmaceutical composition of claim 2, wherein said unit dosage form comprises a salt of octanoic acid.

7. The pharmaceutical composition of claim 6, wherein said salt is an alkali metal salt, an alkaline earth salt, or a basic addition salt.

8. The pharmaceutical composition of claim 1, wherein said unit dosage form is a tablet, pill, capsule, or caplet.

9. The pharmaceutical composition of claim 1, wherein said octanoic acid, or a salt or ester thereof, is substantially pure.

10. The pharmaceutical composition of claim 1, further comprising a second agent selected from beta blockers, anticonvulsants, benzodiazepines, or Botulinum toxin type A and/or B.

11. A method of treating an involuntary tremor in a subject in need thereof, said method comprising administering to said subject a pharmaceutical composition comprising octanoic acid, or a salt or ester thereof, in an amount sufficient to treat said involuntary tremor.

12. The method of claim 11, wherein said pharmaceutical composition is administered one, two, or three times daily.

13. The method of claim 11, wherein said pharmaceutical composition is a pharmaceutical composition of any one of claims 1-10.

14-19. (canceled)

20. The method of claim 11, wherein said pharmaceutical composition is formulated as a syrup, or elixir.

21. (canceled)

22. (canceled)

23. The method of claim 11 any of claim 11, wherein said involuntary tremor is essential tremor, drug-induced tremor, or disease-induced tremor.

24. The method of claim 23, wherein said involuntary tremor is drug-induced tremor associated with the use of cysclosporine, antidepressants, amphetamines, antipsychotics, or caffeine.

25. The method of claim 23, wherein said involuntary tremor is disease-induced associated with Parkinson's disease, Multiple Sclerosis, stroke, head injury, Wilson's disease, mercury poisoning, over-active thyroid gland, or liver encephalopathy.

26. A kit comprising (i) a pharmaceutical composition comprising octanoic acid, or a salt or ester thereof, and (ii) instructions for administering said pharmaceutical composition to a subject for the treatment of involuntary tremors.

27. The kit of claim 26, wherein said pharmaceutical composition is a pharmaceutical composition of any one of claims 1-10.

28-38. (canceled)

39. A method of treating an involuntary tremor in a subject in need thereof, said method comprising the steps of (i) diagnosing said subject as having involuntary tremor; and (ii) administering to said subject a pharmaceutical composition comprising octanoic acid, or a salt or ester thereof, in an amount sufficient to treat said involuntary tremor.

40. The method of claim 39, wherein said involuntary tremor is essential tremor, drug-induced tremor, or disease-induced tremor.

41. The method of claim 39, wherein said involuntary tremor is disease-induced associated with Parkinson's disease, Multiple Sclerosis, stroke, head injury, Wilson's disease, mercury poisoning, over-active thyroid gland, or liver encephalopathy.
